(19) United States
(12) Patent Application Publication
(10) Pub. No.: US 2018/0153867 A1
(43) Pub. Date: Jun. 7, 2018

(54) TREATMENT OF PROSTATE CANCER BY CONCOMITANT ADMINISTRATION OF A BROMODOMAIN INHIBITOR AND A SECOND AGENT

(71) Applicant: Gilead Sciences, Inc., Foster City, CA (US)

(72) Inventor: Philip Vitorino, Redwood City, CA (US)

(21) Appl. No.: 15/817,975

(22) Filed: Nov. 20, 2017

Related U.S. Application Data
(60) Provisional application No. 62/430,705, filed on Dec. 6, 2016.

(51) Int. Cl. A61K 31/444; A61K 31/4166; A61K 31/58

(52) U.S. Cl. CPC A61K 31/444; A61K 31/58; A61K 31/4166

(57) ABSTRACT

Methods for treating prostate cancer comprising administering to a subject in need thereof a bromodomain inhibitor concomitantly with a second agent. The second agent may be an androgen receptor antagonist. In some methods, the prostate cancer is castrate-resistant prostate cancer.

### CROSS-REFERENCE TO RELATED APPLICATION

This application claims the benefit of U.S. Application Ser. No. 62/430,705, filed Dec. 6, 2016, which is hereby incorporated by reference in its entirety.

### BACKGROUND

This application relates to the treatment of prostate cancer.

Castrate-resistant prostate cancer is characterized by disease progression despite androgen-deprivation therapy (ADT) and may present as one or any combination of a continuous rise in serum levels of prostate-specific antigen (PSA), progression of pre-existing disease, or appearance of new metastases. Castrate-resistant prostate cancer can present a spectrum of disease ranging from rising PSA levels without metastases or symptoms and despite adt, to metastases and significant debilitation from cancer symptoms. Prognosis is associated with several factors, including performance status, presence of bone pain, extent of disease on bone scan, and serum levels of alkaline phosphatase. Bone metastases will occur in 90% of men with CRPC and can produce significant morbidity, including pain, pathologic fractures, spinal cord compression, and bone marrow failure. Paraneoplastic effects are also common, including anemia, weight loss, fatigue, hypercoagulability, and increased susceptibility to infection.

### SUMMARY

Disclosed herein are methods of treating prostate cancer comprising administering to a subject in need thereof a bromodomain inhibitor concomitantly with a second agent. In some embodiments, the second agent is an agent used in the treatment of prostate cancer. In some embodiments, the second agent is an androgen receptor antagonist. In some embodiments, the second agent is an androgen synthesis inhibitor. This application also relates to methods of inhibiting cell proliferation comprising administering to a subject in need thereof a bromodomain inhibitor concomitantly with a second agent such as an androgen receptor antagonist.

In some aspects, the prostate cancer is castrate-resistant prostate cancer (CRPC), which may also be known as castration-resistant prostate cancer or castration-recurrent prostate cancer. In some aspects, the CRPC is metastatic CRPC, or mCRPC.

In certain embodiments, the bromodomain inhibitor is a compound of Formula (I)

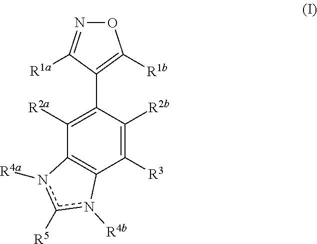

wherein
R^{1a} and R^{1b} are each independently C₁₋₆ alkyl optionally substituted with from 1 to 5 R²⁰ groups;
R^{2a} and R^{2b} are each independently H or halo;
R³ is
   boronic acid or halo; or
   —C(O)OR^{a}, —NHC(O)OR^{a}, —NHS(O)₂R^{a}, or —S(O)₂NR^{a}R^{b}; or
   selected from the group consisting of C₁₋₁₀alkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;
one of R^{4a} and R^{4b} is selected from the group consisting of H and C₁₋₆ alkyl optionally substituted with from 1 to 5 R²⁰ groups, and the other is absent;
R⁵ is
   —C(O)OR^{a}, —NHC(O)OR^{a}, —NHS(O)₂R^{a}, or —S(O)₂NR^{a}R^{b}; or
   selected from the group consisting of H, C₁₋₁₀ alkyl, C₁₋₁₀ haloalkyl, C₁₋₁₀ alkoxy, amino, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups;
each R^{a} and R^{b} is independently selected from the group consisting of H, C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl, each of which is optionally substituted with from 1 to 5 R²⁰ groups; and
each R²⁰ is independently selected from the group consisting of acyl, C₁₋₁₀ alkyl, C₁₋₁₀ alkoxy, amino, amido, amidino, C₅₋₁₀ aryl, C₆₋₂₀ arylalkyl, azido, carbamoyl, carboxyl, carboxyl ester, cyano, guanidino, halo, C₁₋₁₀ haloalkyl, C₁₋₁₀ heteroalkyl, C₅₋₁₀ heteroaryl, C₆₋₂₀ heteroarylalkyl, hydroxy, hydrazino, imino, oxo, nitro, sulfinyl, sulfonic acid, sulfonyl, thiocyanate, thiol, and thione;
   wherein the C₁₋₁₀ alkyl, C₅₋₁₀ aryl, C₆₋₂₀ arylalkyl, C₁₋₁₀ heteroalkyl, C₅₋₁₀ heteroaryl, and C₆₋₂₀ heteroarylalkyl groups are optionally substituted with from 1 to 3 substituents independently selected from C₁₋₆ alkyl, C₅₋₁₀ aryl, halo, C₁₋₆ haloalkyl, cyano, hydroxy, and C₁₋₆ alkoxy;
      or a pharmaceutically acceptable salt or co-crystal or co-crystal thereof.

In certain embodiments, the compound of formula (1) or a pharmaceutically acceptable salt or co-crystal or co-crystal thereof is the compound of formula (I-1)

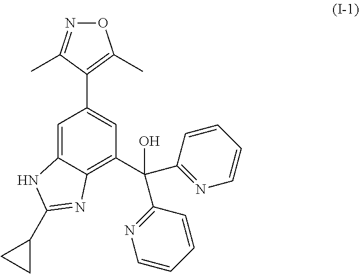 [SMILES: Cc1noc(C)c1-c1cc(C(O)(c2ccccn2)c2ccccn2)c2nc(C3CC3)[nH]c2c1]

or a pharmaceutically acceptable salt or co-crystal thereof. In some embodiments, the pharmaceutically acceptable salt or co-crystal of the compound of formula (I-1) is the phosphate salt or co-crystal. The compound of formula (I-1) is named (2-cyclopropyl-6-(3,5-dimethylisoxazol-4-yl)-1H-benzo[d]imidazol-4-yl)di(pyridin-2-yl)methanol. The phosphate complex of the compound of formula (I-1) (i.e. compound (I-1).H₃PO₄) is named (2-cyclopropyl-6-(3,5-dimethylisoxazol-4-yl)-1H-benzo[d]imidazol-4-yl)di(pyridin-2-yl)methanol phosphate complex or (2-cyclopropyl-6-(3,5-dimethylisoxazol-4-yl)-1H-benzo[d]imidazol-4-yl)di(pyridin-2-yl)methanol phosphate.

### BRIEF DESCRIPTION OF THE FIGURES

FIG. 1 shows the combined effect of compound (I-1) and enzalutamide on cell growth inhibition in VCaP (AR-V7 positive) and LNCaP (AR-V7 negative) cells.

### DETAILED DESCRIPTION

Provided herein are methods for treating a prostate cancer comprising concomitantly administering to a subject in need thereof a compound of formula (I) or a pharmaceutically acceptable salt or co-crystal therof and a second agent. In some embodiments, the second agent is an androgen receptor antagonist or an androgen synthesis inhibitor. In some embodiments, the second agent is an androgen receptor antagonist. In some embodiments, the second agent is an androgen synthesis inhibitor. In some methods, the prostate cancer is castrate-resistant prostate cancer (CRPC), which may also be known as castration-resistant prostate cancer or castration-recurrent prostate cancer. In some methods, the CRPC is metastatic CRPC, or mCRPC. In certain aspects, the androgen receptor antagonist is abiraterone (for example, abiraterone acetate). In other methods, the androgen receptor antagonist is enzalutamide. In other aspects, the androgen receptor antagonist is VT-464. In some methods, the compound of formula (I) or a pharmaceutically acceptable salt or co-crystal therof is the compound of formula (I-1) or a pharmaceutically acceptable salt or co-crystal therof. In some methods, the pharmaceutically acceptable salt or co-crystal of the compound of formula (I-1) is the phosphate salt or co-crystal. In some methods, the subject is a human.

In certain embodiments, the bromodomain inhibitor is selected from the group consisting of

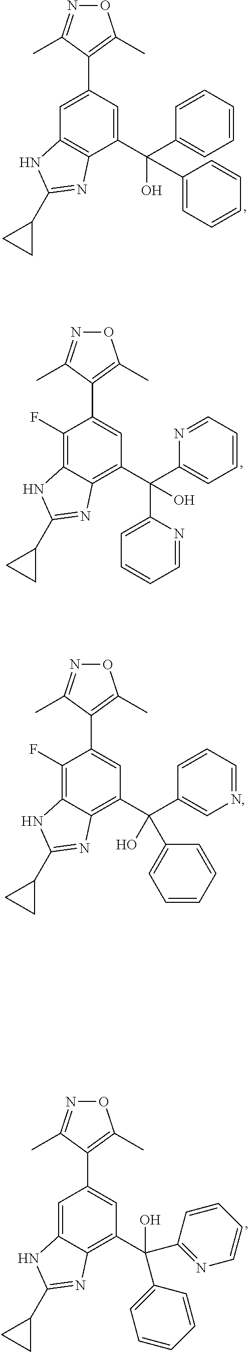

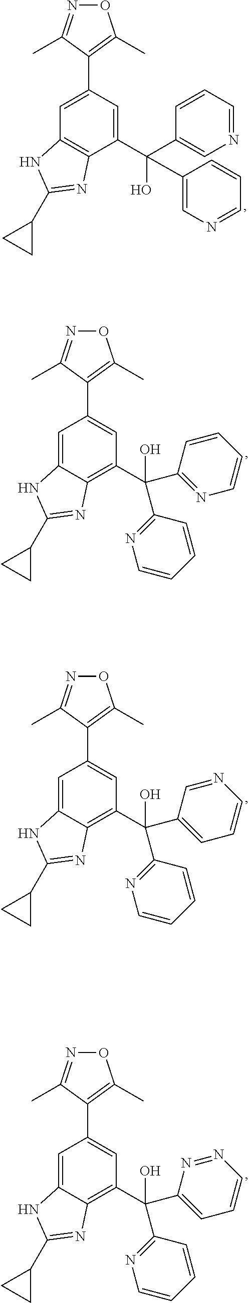

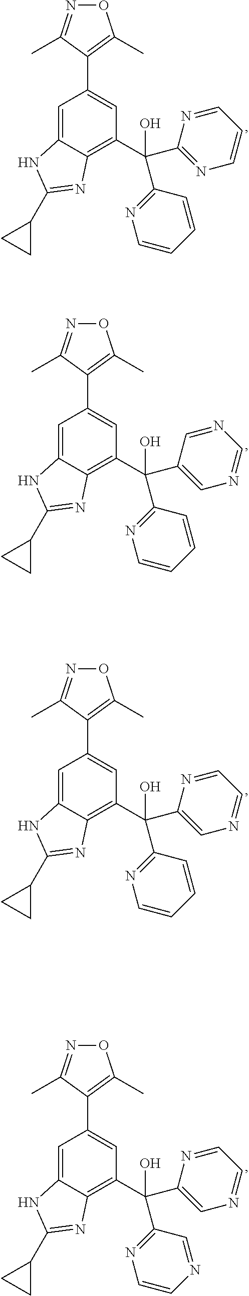

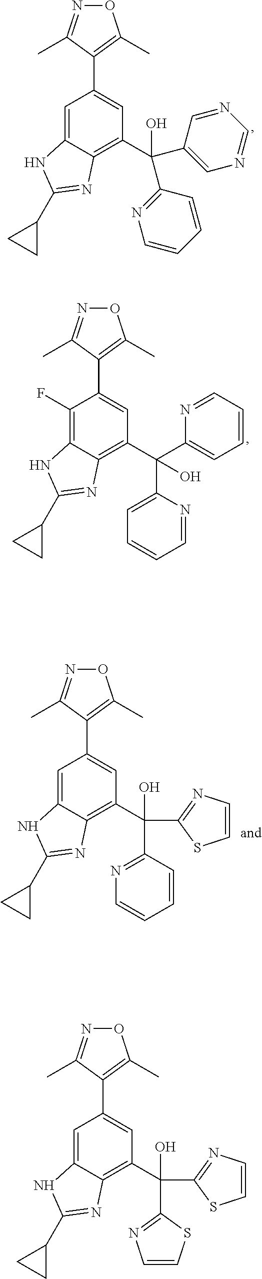

or a pharmaceutically acceptable salt or co-crystal thereof.

In certain embodiments, the bromodomain inhibitor is selected from the group consisting of

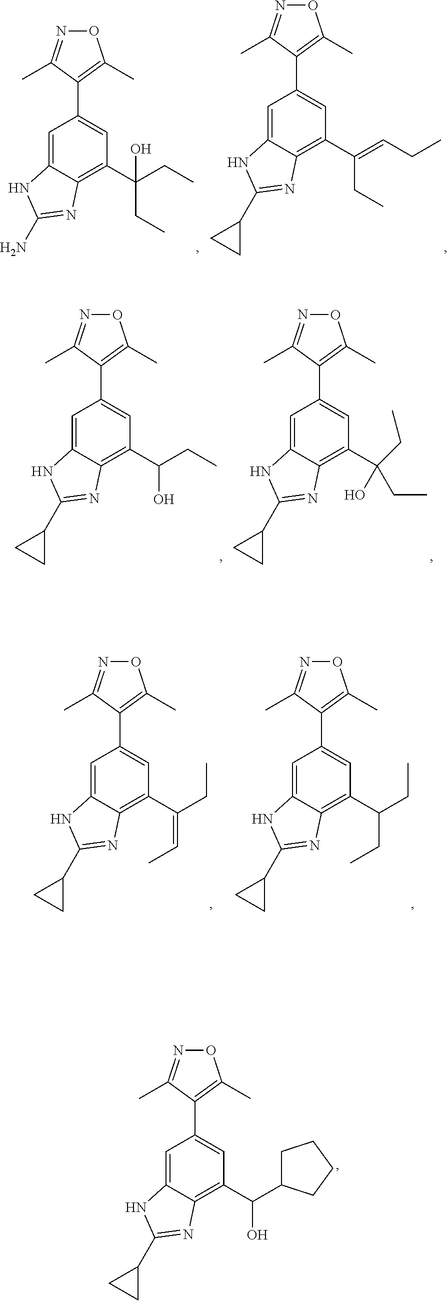

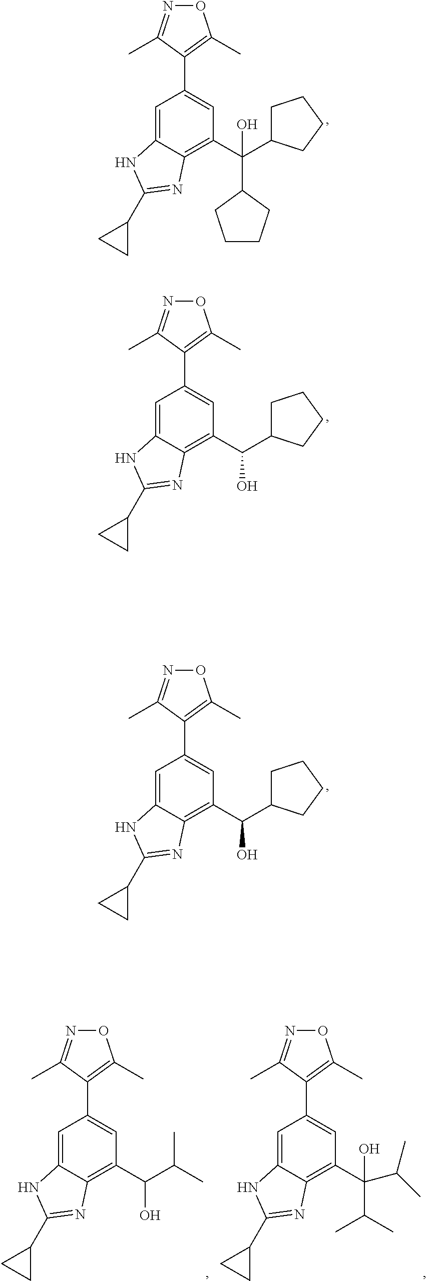 [SMILES: Cc1noc(C)c1-c1cc(C(O)(C2CCCC2)C2CCCC2)c2nc(C3CC3)[nH]c2c1 ; Cc1noc(C)c1-c1cc([C@@H](O)C2CCCC2)c2nc(C3CC3)[nH]c2c1 ; Cc1noc(C)c1-c1cc([C@H](O)C2CCCC2)c2nc(C3CC3)[nH]c2c1 ; Cc1noc(C)c1-c1cc(C(O)C(C)C)c2nc(C3CC3)[nH]c2c1 ; Cc1noc(C)c1-c1cc(C(O)(C(C)C)C(C)C)c2nc(C3CC3)[nH]c2c1]

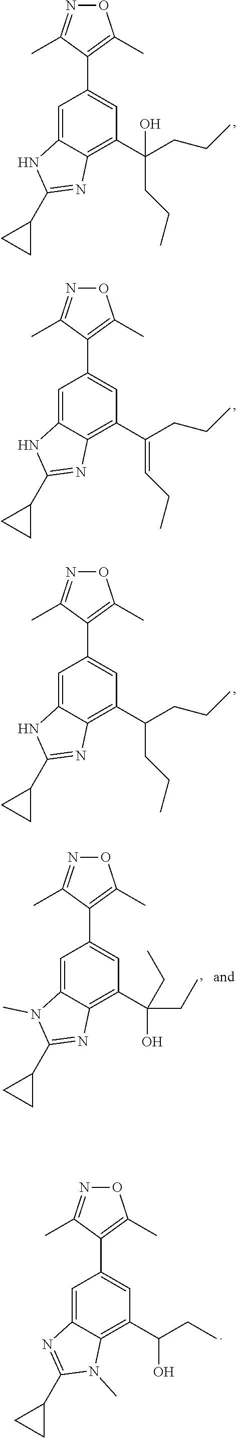

In certain embodiments, the bromodomain inhibitor is selected from the group consisting of

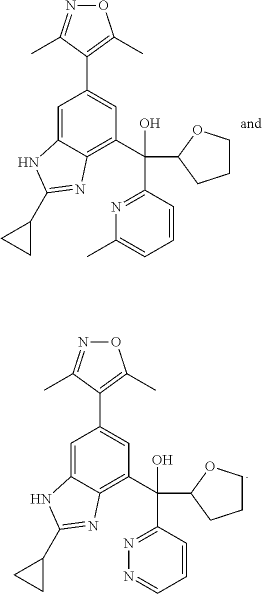 [SMILES: Cc1cccc(C(O)(c2cc(-c3c(C)noc3C)cc3[nH]c(C4CC4)nc23)C2CCCO2)n1 ; "and" ; Cc1noc(C)c1-c1cc(C(O)(c2cccnn2)C2CCCO2)c2nc(C3CC3)[nH]c2c1]

In certain embodiments, the bromodomain inhibitor is selected from the group consisting of

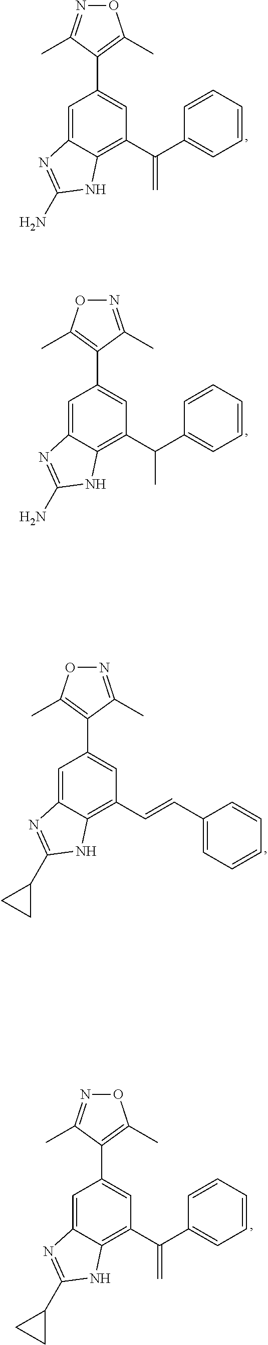 [SMILES: C=C(c1ccccc1)c1cc(-c2c(C)noc2C)cc2nc(N)[nH]c12 ; Cc1noc(C)c1-c1cc(C(C)c2ccccc2)c2[nH]c(N)nc2c1 ; Cc1noc(C)c1-c1cc(/C=C/c2ccccc2)c2[nH]c(C3CC3)nc2c1 ; C=C(c1ccccc1)c1cc(-c2c(C)noc2C)cc2nc(C3CC3)[nH]c12]

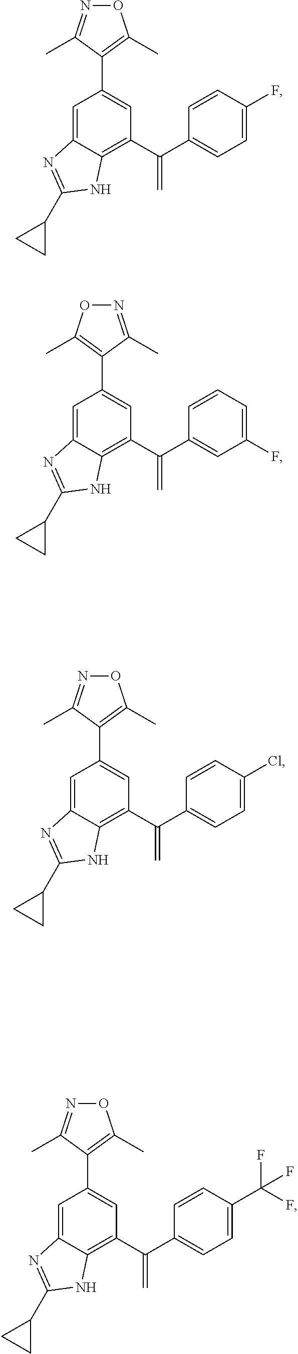 [SMILES: C=C(c1ccc(F)cc1)c1cc(-c2c(C)noc2C)cc2nc(C3CC3)[nH]c12 ; C=C(c1cccc(F)c1)c1cc(-c2c(C)noc2C)cc2nc(C3CC3)[nH]c12 ; C=C(c1ccc(Cl)cc1)c1cc(-c2c(C)noc2C)cc2nc(C3CC3)[nH]c12 ; C=C(c1ccc(C(F)(F)F)cc1)c1cc(-c2c(C)noc2C)cc2nc(C3CC3)[nH]c12]

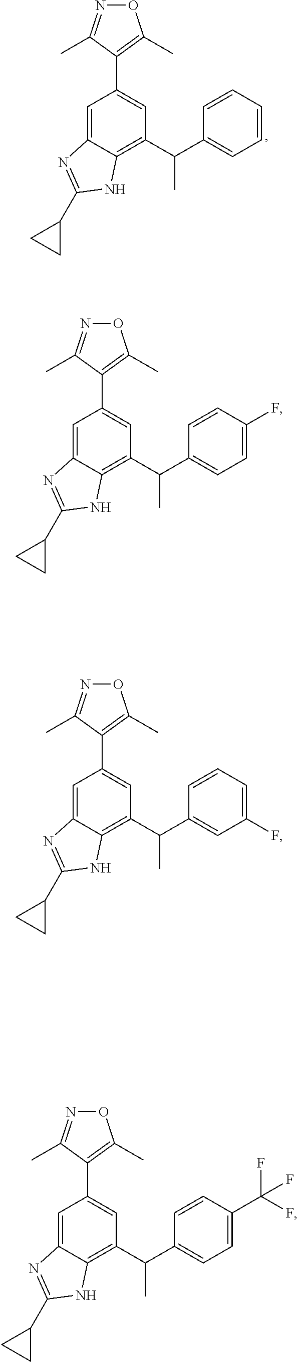 [SMILES: Cc1noc(C)c1-c1cc(C(C)c2ccccc2)c2[nH]c(C3CC3)nc2c1 ; Cc1noc(C)c1-c1cc(C(C)c2ccc(F)cc2)c2[nH]c(C3CC3)nc2c1 ; Cc1noc(C)c1-c1cc(C(C)c2cccc(F)c2)c2[nH]c(C3CC3)nc2c1 ; Cc1noc(C)c1-c1cc(C(C)c2ccc(C(F)(F)F)cc2)c2[nH]c(C3CC3)nc2c1]

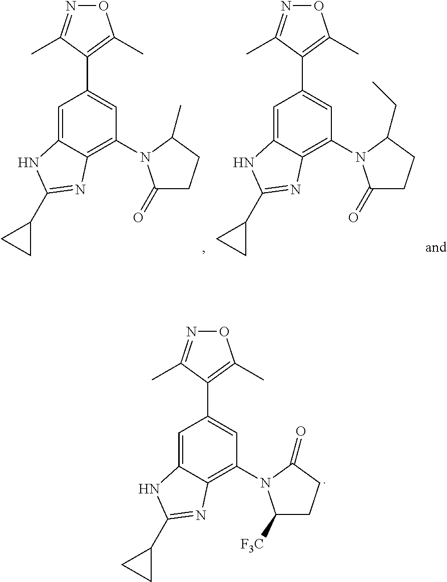 [SMILES: Cc1noc(C)c1-c1cc(N2C(=O)CCC2C)c2nc(C3CC3)[nH]c2c1 ; CCC1CCC(=O)N1c1cc(-c2c(C)noc2C)cc2[nH]c(C3CC3)nc12 ; Cc1noc(C)c1-c1cc(N2C(=O)CC[C@H]2C(F)(F)F)c2nc(C3CC3)[nH]c2c1]

In certain embodiments, the bromodomain inhibitor is selected from the group consisting of

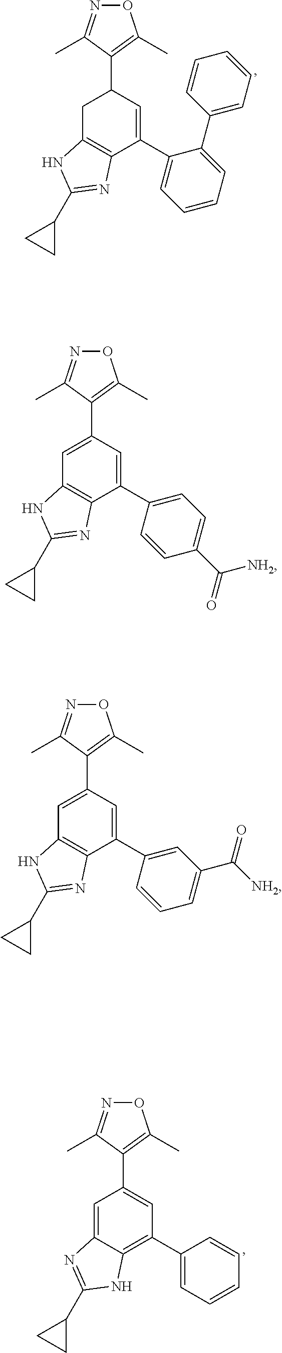 [SMILES: Cc1noc(C)c1C1C=C(c2ccccc2-c2ccccc2)c2nc(C3CC3)[nH]c2C1 ; Cc1noc(C)c1-c1cc(-c2ccc(C(N)=O)cc2)c2nc(C3CC3)[nH]c2c1 ; Cc1noc(C)c1-c1cc(-c2cccc(C(N)=O)c2)c2nc(C3CC3)[nH]c2c1 ; Cc1noc(C)c1-c1cc(-c2ccccc2)c2[nH]c(C3CC3)nc2c1]

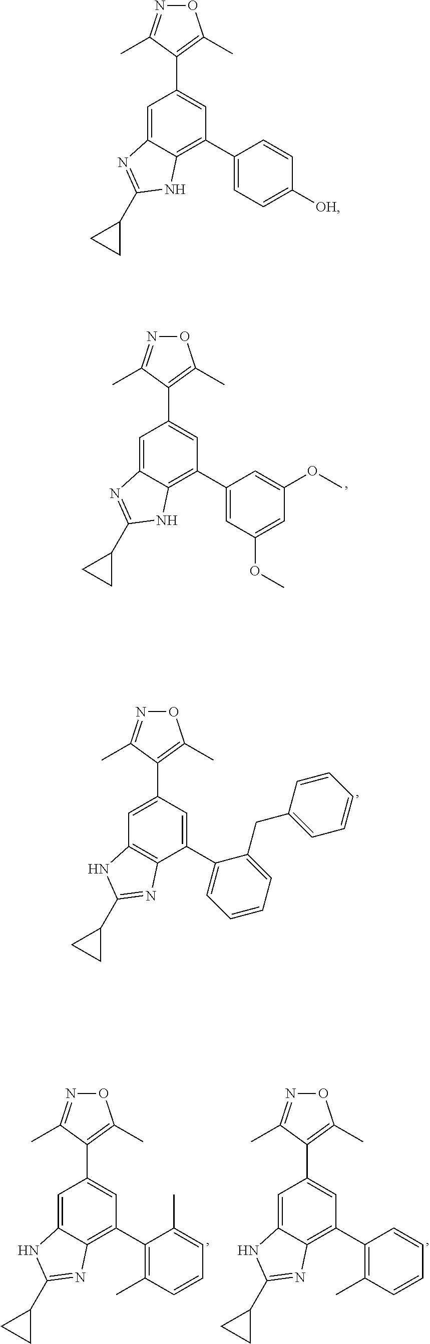 [SMILES: Cc1noc(C)c1-c1cc(-c2ccc(O)cc2)c2[nH]c(C3CC3)nc2c1 ; COc1cc(OC)cc(-c2cc(-c3c(C)noc3C)cc3nc(C4CC4)[nH]c23)c1 ; Cc1noc(C)c1-c1cc(-c2ccccc2Cc2ccccc2)c2nc(C3CC3)[nH]c2c1 ; Cc1cccc(C)c1-c1cc(-c2c(C)noc2C)cc2[nH]c(C3CC3)nc12 ; Cc1ccccc1-c1cc(-c2c(C)noc2C)cc2[nH]c(C3CC3)nc12]

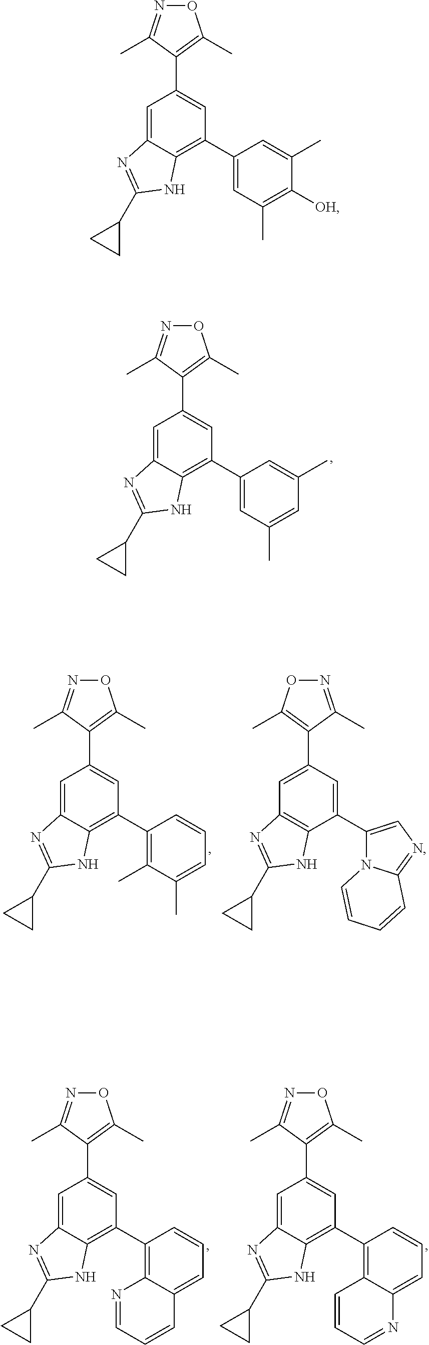

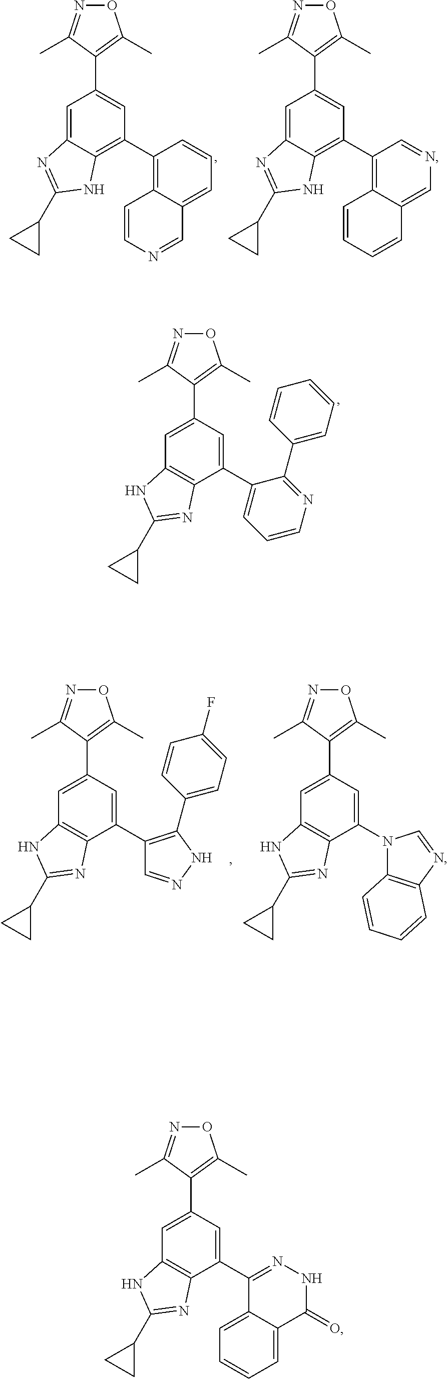

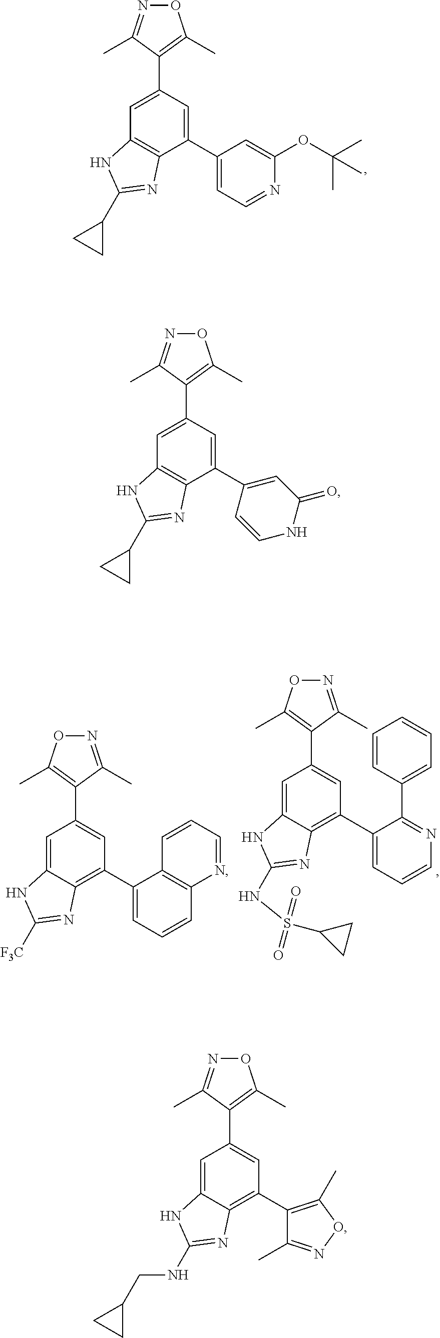

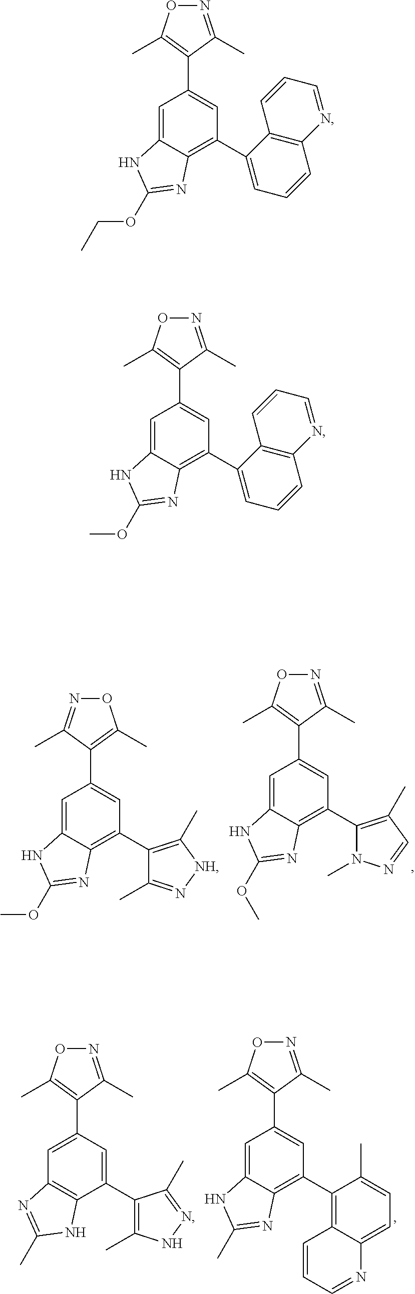

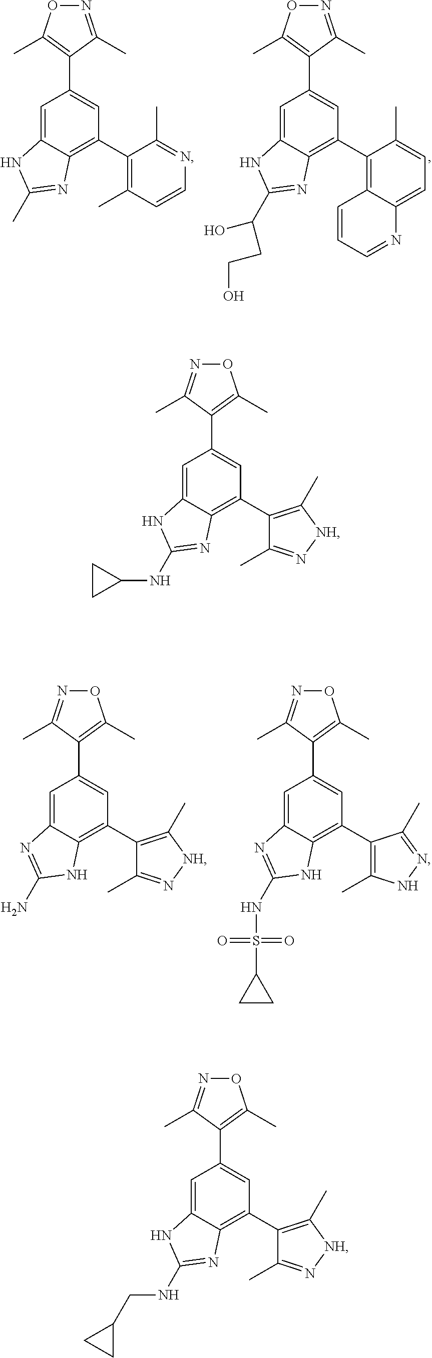 [SMILES: Cc1nc2c(-c3c(C)ccnc3C)cc(-c3c(C)noc3C)cc2[nH]1 ; Cc1ccc2ncccc2c1-c1cc(-c2c(C)noc2C)cc2[nH]c(C(O)CCO)nc12 ; Cc1n[nH]c(C)c1-c1cc(-c2c(C)noc2C)cc2[nH]c(NC3CC3)nc12 ; Cc1n[nH]c(C)c1-c1cc(-c2c(C)noc2C)cc2nc(N)[nH]c12 ; Cc1n[nH]c(C)c1-c1cc(-c2c(C)noc2C)cc2nc(NS(=O)(=O)C3CC3)[nH]c12 ; Cc1n[nH]c(C)c1-c1cc(-c2c(C)noc2C)cc2[nH]c(NCC3CC3)nc12]

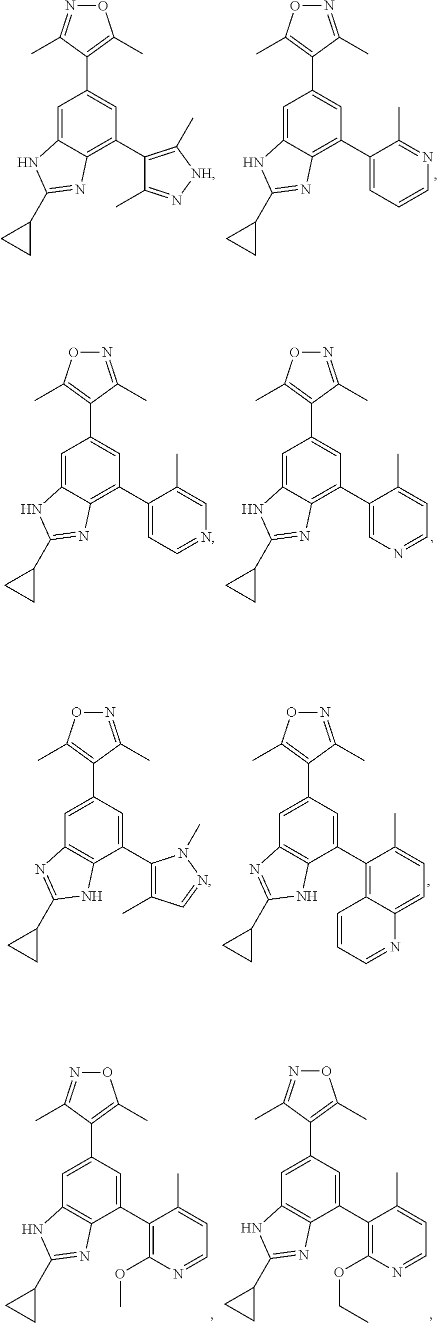

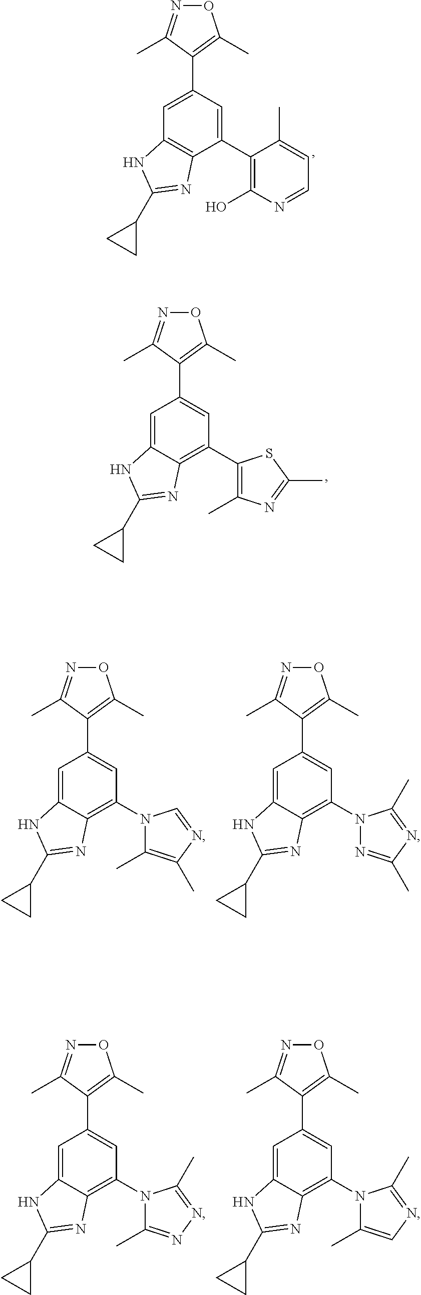

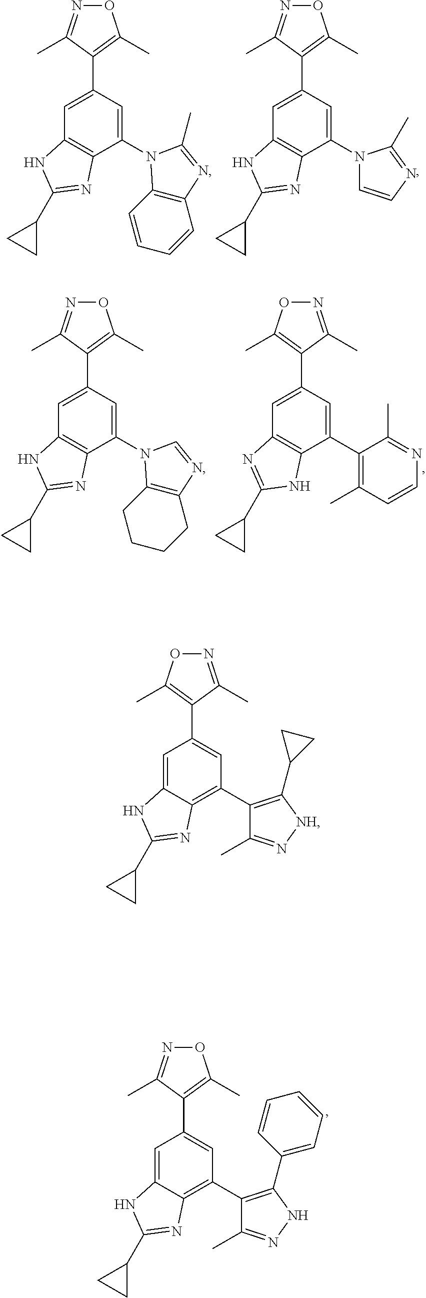

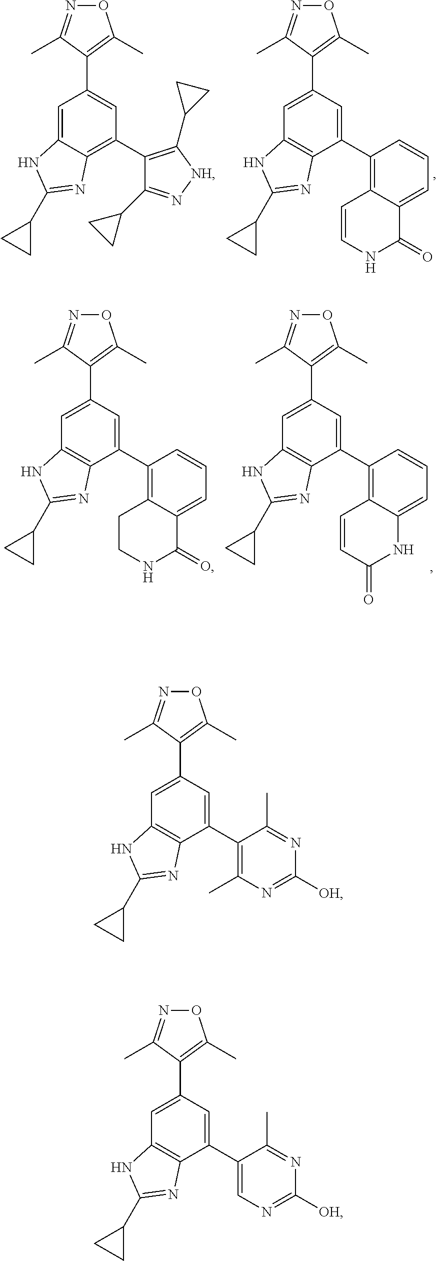

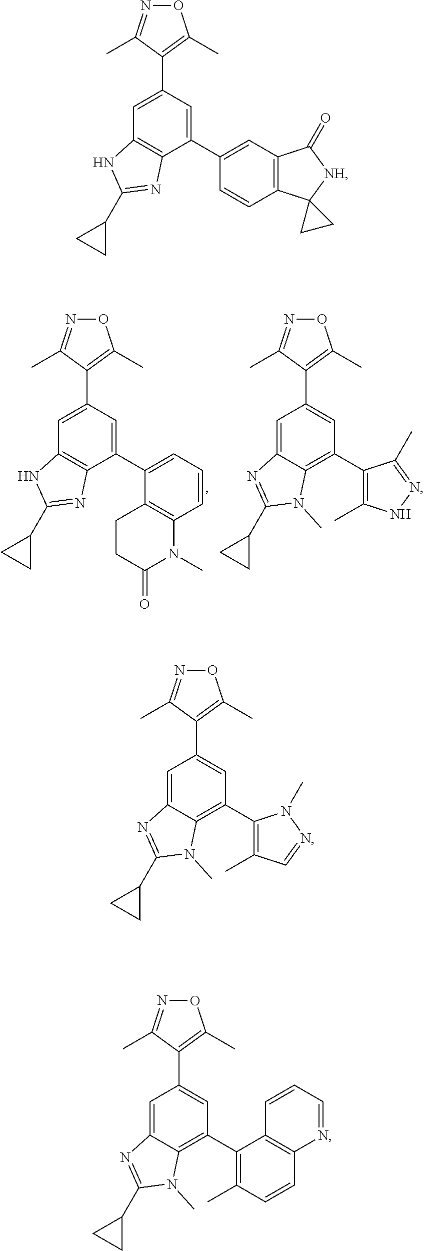

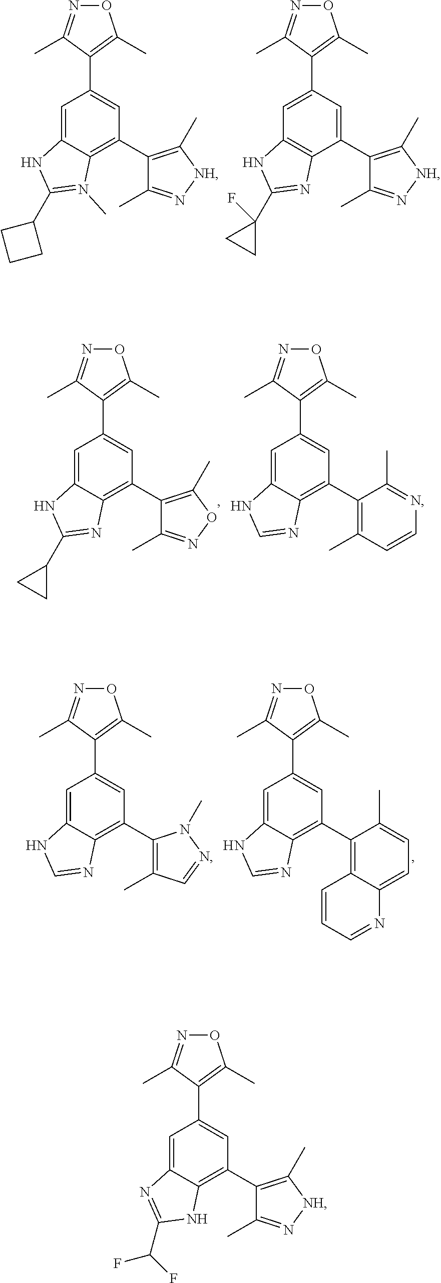

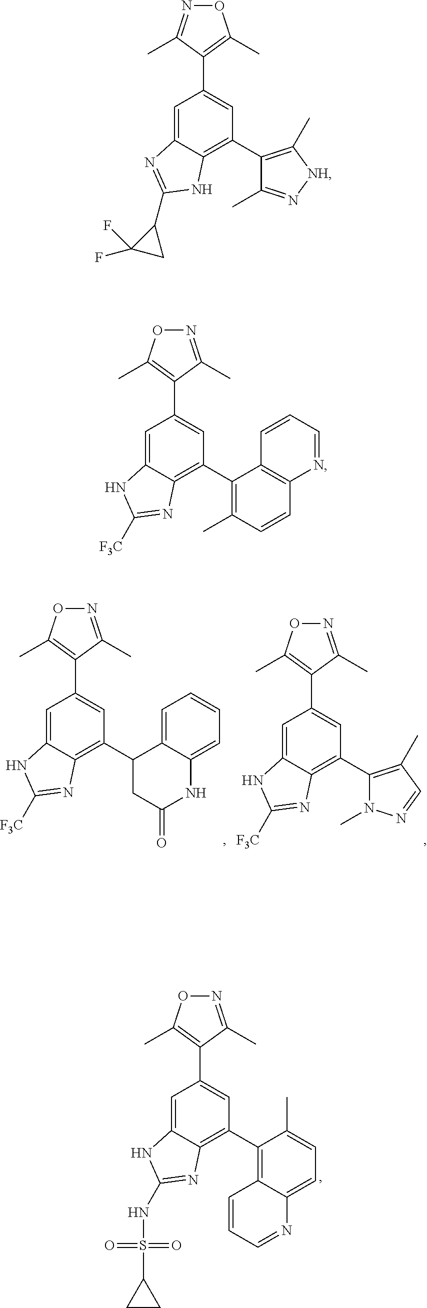 [SMILES: Cc1n[nH]c(C)c1-c1cc(-c2c(C)noc2C)cc2nc(C3CC3(F)F)[nH]c12 ; Cc1ccc2ncccc2c1-c1cc(-c2c(C)noc2C)cc2[nH]c(C(F)(F)F)nc12 ; Cc1noc(C)c1-c1cc(C2CC(=O)Nc3ccccc32)c2nc(C(F)(F)F)[nH]c2c1 ; Cc1cnn(C)c1-c1cc(-c2c(C)noc2C)cc2[nH]c(C(F)(F)F)nc12 ; Cc1ccc2ncccc2c1-c1cc(-c2c(C)noc2C)cc2[nH]c(NS(=O)(=O)C3CC3)nc12]

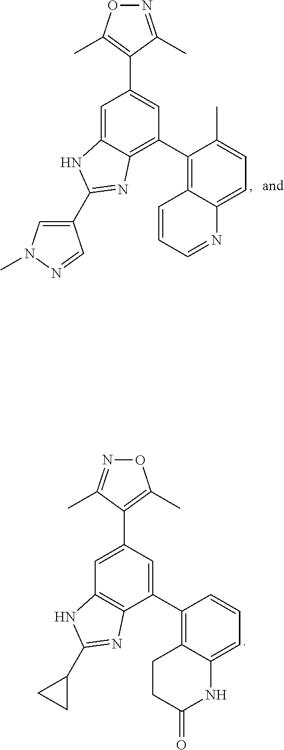 [SMILES: Cc1ccc2ncccc2c1-c1cc(-c2c(C)noc2C)cc2[nH]c(-c3cnn(C)c3)nc12 ; Cc1noc(C)c1-c1cc(-c2cccc3c2CCC(=O)N3)c2nc(C3CC3)[nH]c2c1]

Unless defined otherwise, all technical and scientific terms used herein have the same meaning as commonly understood by one of ordinary skill in the art. It must be noted that as used herein and in the appended claims, the singular forms “a”, “and”, and “the” include plural referents unless the context clearly dictates otherwise. Thus, e.g., reference to “the compound” includes a plurality of such compounds and reference to “the assay” includes reference to one or more assays and equivalents thereof known to those skilled in the art, and so forth.

Unless chemically or structurally required, no directionality is indicated or implied by the order in which a chemical group is written. For instance, the group “—SO₂CH₂—” is equivalent to “—CH₂SO₂—” and both may be connected in either direction. The prefix “Cᵤ₋ᵥ” indicates that the following group has from u to v carbon atoms, one or more of which, in certain groups (e.g. heteroalkyl, heteroaryl, heteroarylalkyl, etc.), may be replaced with one or more heteroatoms or heteroatomic groups. For example, “C₁₋₆ alkyl” indicates that the alkyl group has from 1 to 6 carbon atoms.

Also, certain commonly used alternative chemical names may or may not be used. For example, a divalent group such as a divalent “alkyl” group, a divalent “aryl” group, etc., may also be referred to as an “alkylene” group or an “alkylenyl” group, an “arylene” group or an “arylenyl” group, respectively.

“Alkyl” refers to any aliphatic hydrocarbon group, i.e. any linear, branched, cyclic, or spiro nonaromatic hydrocarbon group or an isomer or combination thereof. As used herein, the term “alkyl” includes terms used in the art to describe saturated and unsaturated aliphatic hydrocarbon groups with one or more points of attachment, including alkenyl (an aliphatic group containing at least one carbon-carbon double bond), alkylene (a divalent aliphatic group), alkynyl (an aliphatic group containing at least one carbon-carbon triple bond), cycloalkyl (a cyclic aliphatic group), alkylcycloalkyl (a linear or branched aliphatic group attached to a cyclic aliphatic group), and the like. Alkyl groups include, but are not limited to, methyl; ethyl; propyls such as propan-1-yl, propan-2-yl (iso-propyl), and cyclopropyls such as cyclopropan-1-yl, etc.; butyls such as butan-1-yl, butan-2-yl (sec-butyl), 2-methyl-propan-1-yl (iso-butyl), 2-methyl-propan-2-yl (t-butyl), cyclobutan-1-yl; butenes (e.g. (E)-but-2-ene, (Z)-but-2-ene); pentyls; pentenes; hexyls; hexenes; octyls; decyls; cyclopropyl, cyclobutyl, cyclopentyl, cyclohexyl, methylcyclohexyl, spiro[2.4]heptyl, and the like. An alkyl group comprises from 1 to about 10 carbon atoms, e.g., from 1 to 6 carbon atoms. In some embodiments, alkyl is a monovalent, linear or branched, saturated aliphatic hydrocarbon group comprising from 1 to about 10 carbon atoms, e.g., from 1 to 6 carbon atoms.

“Alkenyl” is a subset of“alkyl” and refers to an aliphatic group containing at least one carbon-carbon double bond and having from 2 to about 10 carbon atoms, e.g., from 2 to 6 carbon atoms or 2 to 4 carbon atoms and having at least one site of vinyl unsaturation (>C═C<). Alkenyl groups include ethenyl, propenyl, 1,3-butadienyl, and the like. Alkynyl may have from 2 to about 10 carbon atoms, e.g. from 2 to 6 carbon atoms or 2 to 4 carbon atoms.

“Alkynyl” is a subset of“alkyl” and refers to an aliphatic group containing at least one carbon-carbon triple bond. The term “alkynyl” is also meant to include those groups having one triple bond and one double bond.

“Alkoxy” refers to the group —O-alkyl, wherein the alkyl group may be optionally substituted. Alkoxy includes, by way of example, methoxy, ethoxy, n-propoxy, isopropoxy, n-butoxy, t-butoxy, sec-butoxy, and n-pentoxy.

“Acyl” refers to a group —C(═O)R, where R is hydrogen, alkyl, cycloalkyl, cycloheteroalkyl, aryl, arylalkyl, heteroalkyl, heteroaryl or heteroarylalkyl as defined herein, each of which may be optionally substituted, as defined herein. Representative examples include, but are not limited to formyl, acetyl, cylcohexylcarbonyl, cyclohexylmethyl-carbonyl, benzoyl, benzyloxycarbonyl and the like.

“Amido” refers to both a “C-amido” group which refers to the group —C(═O)NR^{y}R^{z} and an “N-amido” group which refers to the group —NR^{y}C(═O)R^{z}, wherein R^{y} and R^{z} are independently selected from the group consisting of hydrogen, alkyl, aryl, heteralkyl, heteroaryl (each of which may be optionally substituted), and where R^{y} and R^{z} are optionally joined together with the nitrogen or carbon bound thereto to form an optionally substituted heterocycloalkyl.

“Amino” refers to the group —NR^{y}R^{z} wherein R^{y} and R^{z} are independently selected from the group consisting of hydrogen, alkyl, aryl, heteralkyl, heteroaryl (each of which may be optionally substituted), and where R^{y} and R^{z} are optionally joined together with the nitrogen bound thereto to form a heterocycloalkyl or heteroaryl heteroaryl (each of which may be optionally substituted).

“Amidino” refers to the group —C(═NR^{x})NR^{y}R^{z} where R^{x}, R^{y}, and R^{z} are independently selected from the group consisting of hydrogen, alkyl, aryl, heteralkyl, heteroaryl (each of which may be optionally substituted), and where R^{y} and R^{z} are optionally joined together with the nitrogen bound thereto to form a heterocycloalkyl or heteroaryl (each of which may be optionally substituted).

“Aryl” refers to a group with one or more aromatic rings. It may be a single aromatic ring or multiple aromatic rings which are fused together, linked covalently, or linked via one or more such as a methylene or ethylene moiety. Aryl groups include, but are not limited to, those groups derived from acenaphthylene, anthracene, azulene, benzene, biphenyl, chrysene, cyclopentadienyl anion, diphenylmethyl, fluoranthene, fluorene, indane, indene, naphthalene, perylene, phenalene, phenanthrene, pyrene, triphenylene, and the like. An aryl group comprises from 5 to about 20 carbon atoms, e.g., from 5 to 20 carbon atoms, e.g. from 5 to 10 carbon atoms. In some embodiments, aryl is a single aromatic ring or multiple aromatic rings which are fused together.

“Arylalkyl” (also “aralkyl”) refers to an aryl group attached to an alkyl group. Arylalkyl groups include, but are not limited to, benzyl, tolyl, dimethylphenyl, 2-phenylethan-1-yl, 2-naphthylmethyl, 2-naphthylethan-1-yl, naphthobenzyl, phenylvinyl, diphenylmethyl, and the like. For example, the “arylalkyl” may be attached to the rest of the compound of formula (I) through the aryl group. Alternatively, the “arylalkyl” may be attached to the rest of the compound of formula (I) through the alkyl group. Where specific alkyl moieties are intended, the nomenclature arylalkanyl, arylalkenyl and/or arylalkynyl may be used. An arylalkyl group comprises from 6 to about 30 carbon atoms, e.g. the alkyl portion of the arylalkyl group can comprise from 1 to about 10 carbon atoms and the aryl portion of the arylalkyl group can comprise from 5 to about 20 carbon atoms. In some instances an arylalkyl group comprises from 6 to about 20 carbon atoms, e.g. the alkyl portion of the arylalkyl group can comprise from 1 to about 10 carbon atoms and the aryl portion of the arylalkyl group can comprise from 5 to about 10 carbon atoms.

“Aryloxy” refers to the group —O-aryl, including by way of example, phenoxy and naphthoxy.

“Azido” refers to the group —N₃.

“Boronic acid” refers to the group —B(OH)₂.

“Boronic acid ester” refers to an ester derivative of a boronic acid compound. Suitable boronic acid ester derivatives include those of the formula —B(OR)₂ where R is hydrogen, alkyl, aryl, arylalkyl, heteroalkyl, or heteroaryl, each of which may be optionally substituted. For example, boronic acid ester may be pinacol ester or catechol ester.

“Carbamoyl” refers to the group —C(O)NR^{y}R^{z} where R^{y} and R^{z} are defined as in “amino” above.

“Carbonyl” refers to the divalent group —C(O)— which is equivalent to —C(═O)—.

“Carboxyl” or “carboxy” refers to —COOH or salt or co-crystals thereof.

“Carboxyl ester” or “carboxy ester” refers to the groups —C(O)OR, wherein R is hydrogen, alkyl, aryl, arylalkyl, heteroalkyl, or heteroaryl, each of which may be optionally substituted. In one embodiment, R is alkyl, aryl, arylalkyl, heteroalkyl, or heteroaryl, each of which may be optionally substituted.

“Cyano” or “carbonitrile” refers to the group —CN.

“Cycloalkyl” is a subset of“alkyl” and refers to a saturated or partially saturated cyclic group of from 3 to about 10 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl.

“Guanidino” refers to the group —NHC(═NH)NH₂.

“Halo” or “halogen” refers to fluoro, chloro, bromo and iodo.

“Haloalkyl” refers to substitution of alkyl groups with 1 to 5 or, in some embodiments, 1 to 3 halo groups, e.g., —CH₂Cl, —CH₂F, —CH₂Br, —CFClBr, —CH₂CH₂Cl, —CH₂CH₂F, —CF₃, —CH₂CF₃, —CH₂CCl₃, and the like, and further includes those alkyl groups such as perfluoroalkyl in which all hydrogen atoms are replaced by fluorine atoms.

“Haloaryl” refers to aryl groups with one or more halo or halogen substituents. For example, haloaryl groups include phenyl groups in which from 1 to 5 hydrogens are replaced with a halogen. Haloaryl groups include, for example, fluorophenyl, difluorophenyl, trifluorophenyl, chlorophenyl, clorofluorophenyl, and the like.

“Heteroalkyl” refers to an alkyl group in which one or more of the carbon atoms (and any associated hydrogen atoms) are each independently replaced with the same or different heteroatom or heteroatomic group. For example, heteroalkyl may include 1, 2 or 3 heteroatomic groups, e.g. 1 heteroatomic group. Heteroatoms include, but are not limited to, N, P, O, S, etc. Heteroatomic groups include, but are not limited to, —NR—, —O—, —S—, —PH—, —P(O)₂—, —S(O)—, —S(O)₂—, and the like, where R is H, alkyl, aryl, cycloalkyl, heteroalkyl, heteroaryl or cycloheteroalkyl. The term “heteroalkyl” includes heterocycloalkyl (a cyclic heteroalkyl group), alkyl-heterocycloalkyl (a linear or branched aliphatic group attached to a cyclic heteroalkyl group), and the like. Heteroalkyl groups include, but are not limited to, —OCH₃, —CH₂OCH₃, —SCH₃, —CH₂SCH₃, —NRCH₃, —CH₂NRCH₃, and the like, where R is hydrogen, alkyl, aryl, arylalkyl, heteroalkyl, or heteroaryl, each of which may be optionally substituted. A heteroalkyl group comprises from 1 to about 10 carbon and hetero atoms, e.g., from 1 to 6 carbon and hetero atoms.

“Heteroaryl” refers to an aryl group in which one or more of the carbon atoms (and any associated hydrogen atoms) are each independently replaced with the same or different heteroatoms, as defined above. For example, heteroaryl may include 1, 2 or 3 heteroatomic groups, e.g. 1 heteroatomic group. Heteroaryl groups include, but are not limited to, groups derived from acridine, benzoimidazole, benzothiophene, benzofuran, benzoxazole, benzothiazole, carbazole, carboline, cinnoline, furan, imidazole, imidazopyridine, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. A heteroaryl group comprises from 5 to about 20 carbon and hetero atoms in the ring or rings, e.g., from 5 to 20 carbon and hetero atoms, e.g. from 5 to 10 carbon and hetero atoms.

“Heteroarylalkyl” refers to an arylalkyl group in which one or more carbon atoms (and any associated hydrogen atoms) are independently replaced with the same or different heteroatoms, as defined above. For example, heteroarylalkyl may include 1, 2 or 3 heteroatomic groups. Heteroarylalkyl groups include, but are no limited to, groups derived from heteroaryl groups with alkyl substituents (e.g. methylpyridine, dimethylisoxazole, etc.), hydrogenated heteroaryl groups (dihydroquinolines, e.g. 3,4-dihydroquinoline, dihydroisoquinolines, e.g. 1,2-dihydroisoquinoline, dihydroimidazole, tetrahydroimidazole, etc.), isoindoline, isoindolones (e.g. isoindolin-1-one), dihydrophthalazine, quinolinone, spiro[cyclopropane-1,1′-isoindolin]-3′-one, di(pyridin-2-yl)methyl, di(pyridin-3-yl)methyl, di(pyridin-4-yl)methyl, and the like. A heteroarylalkyl group comprises from 6 to about 30 carbon and hetero atoms, for example from 6 to about 20 carbon and hetero atoms.

“Heterocycloalkyl” is a subset of “heteroalkyl” and refers to a saturated or unsaturated cycloalkyl group in which one or more carbon atoms (and any associated hydrogen atoms) are independently replaced with the same or different heteroatom. Heteroatoms include, but are not limited to, N, P, O, S, etc. A heterocycloalkyl group may also contain a charged heteroatom or group, e.g., a quaternized ammonium group such as —N+(R)2- wherein R is alkyl, e.g., methyl, ethyl, etc. Heterocycloalkyl groups include, but are not limited to, groups derived from epoxide, imidazolidine, morpholine, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-bromopiperidine, N-chloropyrrolidine, N-chloropiperidine, an N,N-dialkylpyrrolidinium, such as N,N-dimethylpyrrolidinium, a N,N-dialkylpiperidinium such as N,N-dimethylpiperidium, and the like. The heterocycloalkyl group comprises from 3 to about 10 carbon and hetero atoms in the ring or rings. In some embodiments, heterocycloalkyl includes 1, 2 or 3 heteroatomic groups.

“Hydrazino” refers to the group —NHNH₂.

“Hydroxy” or “hydroxyl” refers to the group —OH.

“Imino” refers to the group —C(═NR)— wherein R is hydrogen, alkyl, aryl, arylalkyl, heteroalkyl, or heteroaryl, each of which may be optionally substituted.

“Nitro” refers to the group —NO₂.

The terms “optional” or “optionally” mean that the subsequently described event or circumstance may but need not occur, and that the description includes instances where the event or circumstance occurs and instances in which it does not.

“Oxide” refers to products resulting from the oxidation of one or more heteroatoms. Examples include N-oxides, sulfoxides, and sulfones.

“Oxo” refers to a double-bonded oxygen (═O). In compounds where an oxo group is bound to an sp² nitrogen atom, an N-oxide is indicated.

“Racemates” refers to a mixture of enantiomers.

“Stereoisomer” or “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Substituted” (as in, e.g., “substituted alkyl”) refers to a group wherein one or more hydrogens have been independently replaced with one or more substituents including, but not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocycloalkyl, hydroxy, hydrazino, hydroxyl, imino, oxo, nitro, sulfinyl, sulfonic acid, sulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to -substituted aryl-(substituted aryl)-substituted aryl. For example, in some embodiments, when a group described above as being “optionally substituted” is substituted, that substituent is itself unsubstituted. Similarly, it is understood that the above definitions are not intended to include impermissible substitution patterns (e.g., methyl substituted with 5 fluoro groups or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “arylalkyl.” Generally, substituted groups will have 1 to 5 substituents, 1 to 3 substituents, 1 or 2 substituents or 1 substituent. Alternatively, the optionally substituted groups of the invention may be unsubstituted.

“Sulfonyl” refers to the divalent group —S(O)₂—.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Thiocyanate” refers to the group —SCN.

“Thiol” refers to the group —SH.

“Thione” refers to a thioketone (═S) group.

“Pharmaceutically acceptable” refers to compounds, salt or co-crystals, compositions, dosage forms and other materials which are useful in preparing a pharmaceutical composition that is suitable for veterinary or human pharmaceutical use.

“Pharmaceutically acceptable salt or co-crystal” refers to a salt or co-crystal of a compound that is pharmaceutically acceptable and that possesses (or can be converted to a form that possesses) the desired pharmacological activity of the parent compound. Such salt or co-crystals include acid addition salt or co-crystals formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethylacetic acid, and the like, and salt or co-crystals formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salt or co-crystals. Representative non-limiting lists of pharmaceutically acceptable salt or co-crystals can be found in S. M. Berge et al., J. Pharma Sci., 66(1), 1-19 (1977), and Remington: The Science and Practice of Pharmacy, R. Hendrickson, ed., 21st edition, Lippincott, Williams & Wilkins, Philadelphia, Pa., (2005), at p. 732, Table 38-5, both of which are hereby incorporated by reference herein.

The term “co-crystal” as used herein refers to a single-phase crystalline material of two or more different atoms, ions or molecules. Examples of co-crystals include anhydrates, hydrates, solvates, and clathrates. The components of a co-crystal typically associate via one or more non-covalent interactions such as hydrogen bonding, ionic interactions, van der Waals interactions, and pi-pi interactions. In certain embodiments, the co-crystal of a particular compound can have an improved property as compared to the free form of that compound. In various embodiments, the improved property may include increased solubility, increased dissolution, increased bioavailability, increased dose response, decreased hygroscopicity, a crystalline form of a normally amorphous compound, a crystalline form of a difficult to salt or unsaltable compound, decreased form diversity, or more desired morphology.

The term “complex” as used herein with reference to a compound described herein (e.g. Compound I as a “phosphate complex”) includes a co-crystal and a salt comprising that compound. It should be noted that the difference between a co-crystal and a salt lies merely in the transfer of a proton. The transfer of protons from one component to another in a crystal is dependent on the environment. For this reason, crystalline co-crystals and salts may be thought of as two ends of a proton-transfer spectrum, where an absence of proton transfer exists for co-crystals at one end and where proton transfer has occurred in a salt at the other end.

It understood that combinations of chemical groups may be used and will be recognized by persons of ordinary skill in the art. For instance, the group “hydroxyalkyl” would refer to a hydroxyl group attached to an alkyl group. A great number of such combinations may be readily envisaged.

Compounds of Formula (I) are described further in Application No. PCT/US2014/037344, which is hereby incorporated herewith in its entirety.

“Concomitant administration” refers to the administration of two or more agents (e.g., a bromodomain inhibitor and fulvestrant, or a bromodomain inhibitor and exemestane) in any manner in which the pharmacological effects of those agents are manifested in the subject at the same time. Thus, concomitant administration does not require that a single pharmaceutical composition, the same type of formulation, the same dosage form, or even the same route of administration be used for administration of all of the administered agents, or that the agents be administered at the same time. Concomitant administration may be accomplished by the same dosage form and the same route of administration. One advantage with separate formulations is an added flexibility in dosing, i.e. the dosage of each agent can be changed independently, quickly, and easily. Where separate dosage formulations are used, the agents can be administered at essentially the same time (i.e., simultaneously or concurrently), or at separately staggered times (i.e., sequentially). The agents may also be administered according to separate dosing schedules.

“Effective amount” or “therapeutically effective amount” means the amount of a compound described herein that may be effective to elicit the desired biological or medical response. These terms include the amount of a compound that, when administered to a subject for treating a disease, is sufficient to effect such treatment for the disease. The effective amount will vary depending on the compound, the disease and its severity and the age, weight, etc., of the subject to be treated.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like. In certain embodiments, the subject is a human.

“Treating” and “treatment” of a disease include the following: (1) preventing or reducing the risk of developing the disease, i.e., causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, and (3) relieving the disease, i.e., causing regression of the disease or its clinical symptoms.

In some embodiments, the bromodomain inhibitor is thieno-triazolo-1,4-diazepine (JQ1). In other embodiments, the bromodomain inhibitor is apabetalone (RVX-208), GSK525762, TEN-010, CPI-0610, OTX-015, ZEN-3365, SF2523, SF2535, AU-004, GSK-1210151A, KM601, BGB-3619, and BDOIA298. In other embodiments, the bromodomain inhibitor is a CREBBP inhibitor.

In some embodiments, the second agent is an agent used in the treatment of prostate cancer. Such agents include the following: Abiraterone (Zytiga) and prednisone (multiple brand names); Enzalutamide (Xtandi); Radium-223 (Xofigo) for men with cancer that has spread to the bone; Docetaxel (Docefrez, Taxotere) and prednisone; Sipuleucel-T (Provenge) for men who have few or no symptoms from the cancer; Cabazitaxel (Jevtana) and prednisone for men with prostate cancer that has worsened while receiving docetaxel

In certain aspects, the subject has previously been administered a prostate cancer therapy such as an androgen receptor antagonist or an androgen synthesis inhibitor as monotherapy for treatment of CRPC. The term “monotherapy” means a single active agent is used to treat the medical condition. For example, the subject may have previously been administered abiraterone or enzalutamide.

In one embodiment, the compounds described herein may be administered orally. Oral administration may be via, for example, capsule or enteric coated tablets. The compositions that include at least one compound of Formula (I), or a pharmaceutically acceptable salt or co-crystal thereof, can be formulated so as to provide quick, sustained or delayed release of the active ingredient after administration to the subject by employing procedures known in the art.

The compositions may, in some embodiments, be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, each dosage unit contains from about 1 mg to about 12 mg of a compound of formula (I) or a pharmaceutically acceptable salt or co-crystal thereof. In some embodiments, each dosage unit contains from about 2 mg to about 6 mg of a compound of formula (I) or a pharmaceutically acceptable salt or co-crystal thereof. In some embodiments, each dosage unit contains about 1 mg, about 2 mg, about 3 mg, about 4 mg, about 5 mg, about 6 mg, about 7 mg, about 8 mg, about 9 mg, about 10 mg, about 11 mg or about 12 mg of a compound of formula (I) or a pharmaceutically acceptable salt or co-crystal thereof. In some embodiments, each dosage unit contains about 2 mg of a compound of formula (I) or a pharmaceutically acceptable salt or co-crystal thereof. In some embodiments, each dosage unit contains about 3 mg of a compound of formula (I) or a pharmaceutically acceptable salt or co-crystal thereof. In some embodiments, each dosage unit contains about 4 mg of a compound of formula (I) or a pharmaceutically acceptable salt or co-crystal thereof. In some embodiments, each dosage unit contains about 6 mg of a compound of formula (I) or a pharmaceutically acceptable salt or co-crystal thereof.

In some embodiments, each dosage unit contains from about 1 mg to about 12 mg of a compound of formula (I-1) or a pharmaceutically acceptable salt or co-crystal thereof. In some embodiments, each dosage unit contains from about 2 mg to about 6 mg of a compound of formula (I-1) or a pharmaceutically acceptable salt or co-crystal thereof. In some embodiments, each dosage unit contains about 1 mg, about 2 mg, about 3 mg, about 4 mg, about 5 mg, about 6 mg, about 7 mg, about 8 mg, about 9 mg, about 10 mg, about 11 mg or about 12 mg of a compound of formula (I-1) or a pharmaceutically acceptable salt or co-crystal thereof. In some embodiments, each dosage unit contains about 2 mg of a compound of formula (I-1) or a pharmaceutically acceptable salt or co-crystal thereof. In some embodiments, each dosage unit contains about 3 mg of a compound of formula (I-1) or a pharmaceutically acceptable salt or co-crystal thereof. In some embodiments, each dosage unit contains about 4 mg of a compound of formula (I-1) or a pharmaceutically acceptable salt or co-crystal thereof. In some embodiments, each dosage unit contains about 6 mg of a compound of formula (I-1) or a pharmaceutically acceptable salt or co-crystal thereof.

A compound of formula (I) or a pharmaceutically acceptable salt or co-crystal thereof may be administered to the subject in an amount from about 1 mg to about 12 mg per day. A compound of formula (I) or a pharmaceutically acceptable salt or co-crystal thereof may be administered to the subject in an amount from about 2 mg to about 6 mg per day. In some embodiments, a compound of formula (I) or a pharmaceutically acceptable salt or co-crystal thereof may be administered to the subject in an amount of about 1 mg per day, about 2 mg per day, about 3 mg per day, about 4 mg per day, about 5 mg per day, about 6 mg per day, about 7 mg per day, about 8 mg per day, about 9 mg per day, about 10 mg per day, about 11 mg per day or about 12 mg per day. In some embodiments, a compound of formula (I) or a pharmaceutically acceptable salt or co-crystal thereof may be administered to the subject in an amount of about 2 mg per day. In some embodiments, a compound of formula (I) or a pharmaceutically acceptable salt or co-crystal thereof may be administered to the subject in an amount of about 3 mg per day. In some embodiments, a compound of formula (I) or a pharmaceutically acceptable salt or co-crystal thereof may be administered to the subject in an amount of about 4 mg per day. In some embodiments, a compound of formula (I) or a pharmaceutically acceptable salt or co-crystal thereof may be administered to the subject in an amount of about 6 mg per day.

A compound of formula (I-1) or a pharmaceutically acceptable salt or co-crystal thereof may be administered to the subject in an amount from about 1 mg to about 12 mg per day. A compound of formula (I-1) or a pharmaceutically acceptable salt or co-crystal thereof may be administered to the subject in an amount from about 2 mg to about 6 mg per day. In some embodiments, a compound of formula (I-1) or a pharmaceutically acceptable salt or co-crystal thereof may be administered to the subject in an amount of about 1 mg per day, about 2 mg per day, about 3 mg per day, about 4 mg per day, about 5 mg per day, about 6 mg per day, about 7 mg per day, about 8 mg per day, about 9 mg per day, about 10 mg per day, about 11 mg per day or about 12 mg per day. In some embodiments, a compound of formula (I-1) or a pharmaceutically acceptable salt or co-crystal thereof may be administered to the subject in an amount of about 2 mg per day. In some embodiments, a compound of formula (I-1) or a pharmaceutically acceptable salt or co-crystal thereof may be administered to the subject in an amount of about 3 mg per day. In some embodiments, a compound of formula (I-1) or a pharmaceutically acceptable salt or co-crystal thereof may be administered to the subject in an amount of about 4 mg per day. In some embodiments, a compound of formula (I-1) or a pharmaceutically acceptable salt or co-crystal thereof may be administered to the subject in an amount of about 6 mg per day.

The phosphate salt or co-crystal (i.e. phosphate complex) of formula (I-1) may be administered to the subject in an amount from about 1 mg to about 12 mg per day. The phosphate salt or co-crystal (i.e. phosphate complex) of formula (I-1) may be administered to the subject in an amount from about 2 mg to about 6 mg per day. In some embodiments, the phosphate complex of compound of formula (I-1) may be administered to the subject in an amount of about 1 mg per day, about 2 mg per day, about 3 mg per day, about 4 mg per day, about 5 mg per day, about 6 mg per day, about 7 mg per day, about 8 mg per day, about 9 mg per day, about 10 mg per day, about 11 mg per day or about 12 mg per day. In some embodiments, the phosphate complex of compound of formula (I-1) may be administered to the subject in an amount of about 2 mg per day. In some embodiments, the phosphate complex of compound of formula (I-1) may be administered to the subject in an amount of about 3 mg per day. In some embodiments, the phosphate complex of compound of formula (I-1) may be administered to the subject in an amount of about 4 mg per day. In some embodiments, the phosphate complex of compound of formula (I-1) may be administered to the subject in an amount of about 6 mg per day.

In some embodiments, the compound of formula (I) or a pharmaceutically acceptable salt or co-crystal thereof may be dosed once per day. In some embodiments, the compound of formula (I-1) or a pharmaceutically acceptable salt or co-crystal thereof may be dosed once per day. In other embodiments, the compound of formula (I) or a pharmaceutically acceptable salt or co-crystal thereof including the compound of formula (I-1) or a pharmaceutically acceptable salt or co-crystal thereof may be dosed according to a different dosing schedule, such as twice per day, once every two days, two days “on” one day “off,” and so forth.

In various embodiments, the dosing of the second agent is that of the current proscribing information (i.e. the product insert).

In certain methods, the first administration of a compound of formula (I) or a pharmaceutically acceptable salt or co-crystal thereof may be in one or more different patient segments, as follows:
Segment 1: Asymptomatic non-metastatic patients characterized by rising PSA but scans are negative for measurable disease. Outside the context of clinical trials, no treatment options are currently recommended for non-metastatic CRPC;
Segment 2: Metastatic patients who are abiraterone/enzalutamide therapy naïve and chemo naïve (pre-taxane), characterized by rising PSA and a scan demonstrating metastases;
Segment 3: Metastatic patients who are abiraterone/enzalutamide therapy treated and chemonaïve (pre-taxane), for which retreatment with another androgen receptor-therapy (abiraterone or enzalutamide) is not recommended due to cross-resistance;
Segment 4: Metastatic patients who are abiraterone/enzalutamide therapy treated and chemo treated.

One method provides for the administration of a compound of formula (I-1) or a pharmaceutically acceptable salt or co-crystal thereof in metastatic patients who are abiraterone/enzalutamide therapy naïve and chemo naïve (pre-taxane). One method provides for the administration of a compound of formula (I-1) or a pharmaceutically acceptable salt or co-crystal thereof in metastatic patients who are abiraterone/enzalutamide therapy treated and chemonaïve (pre-taxane), for which retreatment with another androgen receptor-therapy (abiraterone or enzalutamide) is not recommended due to cross-resistance. One method provides for the administration of a compound of formula (I-1) or a pharmaceutically acceptable salt or co-crystal thereof in metastatic patients who are abiraterone/enzalutamide therapy treated and chemo treated.

### Examples

### In Vitro Assays

A combination of compound (I-1) and enzalutamide increases cell growth inhibition in VCaP and LNCaP cells. The combined effect of compound (I-1) and enzalutamide on cell growth inhibition was evaluated in VCaP (AR-V7 positive) and LNCaP (AR-V7 negative) cells. VCaP cells were grown under standard cell culture conditions in 10% fetal bovine serum (FBS). LNCaP cells were grown in charcoal stripped FBS supplemented with 10 nM DHT in a 3-dimensional cell spheroid culture system, which increases androgen-dependent growth. VCaP and LNCaP cells were treated with vehicle or increasing concentration of compound (I-1) (3 to 10,000 nM), enzalutamide (3 to 10,000 nM), or compound (I-1) (3 to 10,000 nM) in the presence of 3,000 nM enzaluatmide. This concentration of enzalutamide (3000 nM) was chosen for the combination study because it matches the clinical steady state trough level of enzalutamide at the recommended dose of 160 mg QD after accounting for differences in protein binding between cell culture media and human plasma. Cell growth and number was measured by quantification of the total cellular ATP content (Cell Titer-Glo® Luminescent Cell Viability Assay) at the time of compound addition (T=0), and after 96 hours of treatment. Cell Titer-Glo values were normalized to background values observed in the absence of cells (0%) and values observed in DMSO (vehicle) treated cells at the end of the assay (100%).

Compound (I-1) potently reduced the growth of VCaP cells and decreased the number of viable cells to below that recorded at the beginning of the assay (T=0), consistent with the reported ability of BET inhibitors to induce apoptosis in VCaP cells. Enzalutamide reduced the growth of VCaP cells but the magnitude of growth suppression was significantly less than that observed for compound (I-1). In the presence of enzalutamide, the activity of compound (I-1) was increased at clinically relevant concentration that span the projected Cmax and Cmin when corrected for protein binding. For example, compound (I-1) inhibited cell growth and viability by 40% and 0% at the projected Cmax and Cmin of compound (I-1), respectively, whereas addition of 3,000 nM enzalutamide with these levels of compound (I-1) inhibited cell growth and viability 65% and 25%, respectively. Thus, the addition of enzalutamide to compound (I-1) had a combination effect in vitro against VCaP prostate cancer cells. In the presence of 3,000 nM enzalutamide, the compound (I-1) EC50 was decreased from 130 nM to 75 nM.

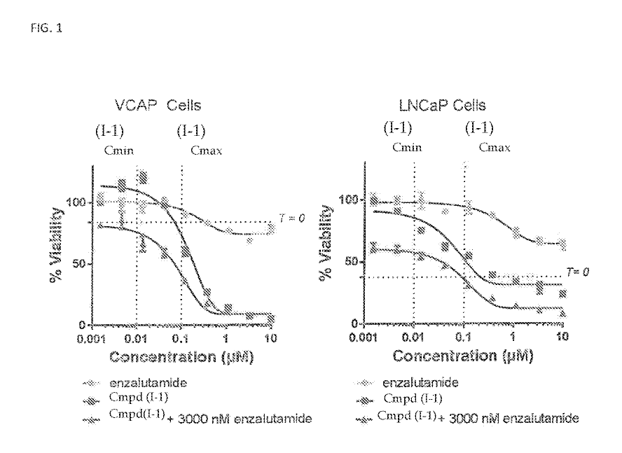

The effect of enzalutamide and compound (I-1) on inhibition of cell growth and viability was also evaluated in LNCaP prostate cancer cells in vitro (FIG. 1). At the projected Cmax and Cmin, compound (I-1) inhibited cell growth and viability by 45% and 20%, respectively. When combined with 3,000 nM enzalutamide, compound (I-1) inhibited cell growth and viability at the projected Cmax and Cmin of compound (I-1) by 65% and 45%, respectively. Thus, the addition of enzalutamide to compound (I-1) increased the magnitude of response in vitro against LNCaP prostate cancer cells.

These in vitro data demonstrating additive effects of compound (I-1) and enzalutamide suggest the potential to increase clinical efficacy by combination of compound (I-1) and enzalutamide in patients with CRCP. In addition, the data suggest that combination with enzalutamide may enable the use of a lower dose of compound (I-1) and thus could increase the therapeutic index of compound (I-1).

Referring again to FIG. 1, the Y-axis is normalized CellTiter-Glo values where the bottom of the assay (no ATP)=0% and the DMSO-treated cells=100% at the end of the assay. T=0 represents the CellTiter-Glo signal recorded at the time of compound addition. The estimated clinical Cmin and Cmax of compound (I-1) at the predicted therapeutic dose of 25 mg are shown (adjusted for the 5.7× difference in protein binding between cell culture media and human plasma). One representative of three independent experiments is shown.

### Clinical Trial

Compound (I-1).H₃PO₄ can be tested in human subjects with CRPC as a single agent and in combination with enzalutamide.

The Phase 1 study will be a 3+3 dose escalation to evaluate the safety, tolerability, PK, and maximum tolerated dose of compound (I-1) after multiple dosing in patients with CRPC. The initial dose will be at 0.6 mg, and intensive safety, PK and PD monitoring will occur prior to each dose escalation. The dose escalation will be limited to an increase of approximately 2× at each subsequent dose level due to the steep dose/toxicity relationship observed in the dog toxicity studies. Approximately six dose levels will be explored. The PD assessment will be a NanoString assay that measures levels of HEXIM1 and CCR2 RNA in peripheral blood cells.

A phase 1b studies can begin to enroll after completion of the 28 day safety observation period in the cohort where clinical efficacy or target coverage (assessed by PD markers in blood cells) is observed. Tolerability, PK, PD and clinical efficacy will be used to determine the dose for the Phase 2 expansion portion of these two studies.

The Dose Expansion study in mCRPC for 2 cohorts can be done as follows: Cohort 1 metastatic patients who are abiraterone/enzalutamide therapy resistant and taxane treated and Cohort 2 metastatic patients who are abiraterone/enzalutamide therapy resistant and taxane naïve. Success criteria for compound (I-1) as monotherapy in the Phase 2 mCRPC studies will be established based on ≥50% of patients receiving benefit from compound (I-1) as measured by a decline in PSA of at least 50% and ≥8 month time to PSA progression/radiographic progression at a dose which is safe and well tolerated. These success criteria are chosen based on similar improvements in PFS and PSA response in enzalutamide which ultimately corresponded to a 3.4 month increase is OS (HR 0.69).

A Phase 3 trial can be a prospective double blind, randomized trial of compound (I-1) compared to placebo in patients with metastatic CRPC who are abiraterone/enzalutamide therapy resistant and taxane treated. The primary endpoint can be overall survival (OS). Assuming a 12 month enrollment and a HR of 0.7, 534 subjects would need to be enrolled (assuming a 10% drop out rate) to demonstrate with 90% power, superiority in OS compared to placebo.

Another Phase 3 study can enroll metastatic patients who are abiraterone/enzalutamide therapy resistant and taxane naïve. Subjects will be randomized in a double blind trial to compound (I-1) or placebo. The primary endpoint will be OS and secondary endpoints will include radiographic PFS and time to initiation of chemotherapy. Assuming a 12 month enrollment and a HR of 0.7, 610 subjects would need to be enrolled (assuming 10% drop out rate) to demonstrate with 90% power, superiority in OS. Stratification by prior response to AR therapy (abiraterone and/or enzalutamide) may be considered. The expression of AR-V7 in circulating tumor cells, as well as other poor prognostic laboratory markers could be collected as an experimental/correlative biomarker, allowing the ability to demonstrate efficacy across all CRPC subgroups.

While the foregoing description describes specific embodiments and aspects, those with ordinary skill in the art will appreciate that various modifications and alternatives can be developed. Accordingly, the particular embodiments and aspects described above are meant to be illustrative only, and not to limit the scope of the invention, which is to be given the full breadth of the appended claims, and any and all equivalents thereof.

We claim:

1. A method for treating prostate cancer comprising concomitantly administering to a subject in need thereof a compound of formula (I-1) 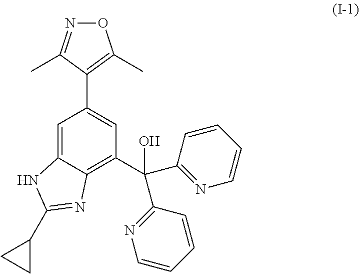 [SMILES: Cc1noc(C)c1-c1cc(C(O)(c2ccccn2)c2ccccn2)c2nc(C3CC3)[nH]c2c1]
or a pharmaceutically acceptable salt or co-crystal thereof, and a second agent, wherein the second agent is an androgen receptor antagonist or an androgen synthesis inhibitor.

2. The method of claim 1, wherein the second agent is enzalutamide.

3. The method of claim 1, wherein the second agent is abiraterone.

4. The method of claim 1, wherein the prostate cancer is castrate-resistant prostate cancer.

5. The method of claim 1, wherein the pharmaceutically acceptable salt or co-crystal is the phosphate salt or co-crystal.

6. The method of claim 1, wherein the subject has previously been administered a prostate cancer therapy.

7. The method of claim 1, wherein the subject is a human.
